(19) United States
(12) Patent Application Publication
(10) Pub. No.: US 2007/0072205 A1
(43) Pub. Date: Mar. 29, 2007

(54) NANOMATERIAL ERROR CORRECTION

(76) Inventors: Yi Lu, Champaign, IL (US); Juewen Liu, Urbana, IL (US); Daryl P. Wernette, Champaign, IL (US)

Correspondence Address: EVAN LAW GROUP LLC, 600 WEST JACKSON BLVD., SUITE 625, CHICAGO, IL, 60661, US

(21) Appl. No.: 11/423,307

(22) Filed: Jun. 9, 2006

Related U.S. Application Data
(60) Provisional application No. 60/688,961, filed on Jun. 9, 2005.

(51) Int. Cl. C12Q 1/68; C12P 19/34; C12N 9/22

(52) U.S. Cl. 435/6; 435/7.4; 435/199; 435/91.2

(57) ABSTRACT

The present invention makes use of the discovery that proofreading and error-correction techniques common in biological systems may be adapted to material science. Enzymes and aptamers are adapted to proofread and correct defects in nanoparticle structures.

### REFERENCE TO RELATED APPLICATIONS

This application claims the benefit of U.S. Provisional Application No. 60/688,961 entitled “Nanomaterial Error Correction” filed Jun. 9, 2005, which is incorporated by reference in its entirety.

### FEDERALLY SPONSORED RESEARCH OR DEVELOPMENT

The subject matter of this application may have been funded in part under the following research grants and contracts: DOD through a Multidisciplinary University Research Initiative (MURI) Program (Grant No. DAAD19-03-1-0227) and National Science Foundation (NSF) through a Nanoscale Science and Engineering Center (NSEC) program (Grant No. DMR-0117792). The U.S. Government may have rights in this invention.

### BACKGROUND

Nanostructures may be self-assembled for many applications, including molecular electronics, photonics, and analyte sensors.¹⁻⁵ Errors and imperfections in self-assembled nanostructures are a significant problem.

Conventional techniques for reducing the errors in self-assembled nanostructures focus on optimizing the assembly process to reduce the errors in the final structure and designing devices that operate effectively with the structural errors.⁶⁻⁹ To reduce assembly errors, conventional methods use time and cost intensive processes that include clean-room processing and the like.

Biological systems deal with structural errors in a different way. Instead of attempting to provide systems that do not create errors, nature employs proofreading and error correction. FIG. 1 represents an eloquent biological example of proofreading and error correction during and after self-assembly in the form of mRNA-templated protein synthesis.¹⁰ In this representation, an incorrect tRNA and its amino acid is incorporated into a protein during self-assembly of the protein on a mRNA template. A proofreading/error-corrector, such as GTPase elongation factor-Tu, removes the error.

Multiple theoretical methods of proofreading and error correction have been described for nanomaterial synthesis, see Winfree, et al., Proofreading Tile Sets: Error Correction for Algorithmic Self-Assembly, Lecture Notes in Computer Science, 2943, 126-144. One experimental technique incorporates a nature-based protein enzyme into a PCR reaction to provide error-free replication of DNA during DNA synthesis. A more detailed description of this technique is found in Nucleic Acids Research, 32, e162, (2004).

It would be beneficial if the proofreading and error correction of biological systems could be adapted to material science. In this manner, the present need to self-assemble perfect nanostructures may be reduced and the errors that result from self-assembly could be corrected.

### SUMMARY

In one aspect, the invention provides a self-assembled nanostructure including appropriate units and at least one error unit, where each unit includes an oligonucleotide and the error correcting unit removes the at least one error unit in response to an effector.

In another aspect, the invention provides a composition for correcting errors in self-assembled nanostructures including means for proofreading self-assembled nanostructures and means for correcting at least one error in the self-assembled nanostructure.

In another aspect, the invention provides a method of removing a first error unit from a self-assembled nanostructure by an error correcting unit cleaving a substrate or folding in response to an effector. The self-assembled nanostructure includes at least a first appropriate unit, a second appropriate unit, and the first error unit, where each unit comprises an oligonucleotide.

The following definitions are included to provide a clear and consistent understanding of the specification and claims.

The term “co-factor” refers to any ion or molecule that can activate an error-correction enzyme. Preferable monovalent metal ions having a ⁺1 formal oxidation state (I) include Li(I), TI(I), and Ag(I). Preferable divalent metal ions having a ⁺2 formal oxidation state (II) include Mg(II), Ca(II), Mn(II), Co(II), Ni(II), Zn(II), Cd(II), Cu(II), Pb(II), Hg(II), Pt(II), Ra(II), Sr(II), Ni(II), and Ba(II). Preferable trivalent and higher metal ions having ⁺3 (III), ⁺4 (IV), ⁺5 (V), or ⁺6 (VI) formal oxidation states include Co(III), Cr(III), Ce(IV), As(V), U(VI), Cr(VI), and lanthanide ions.

The term “hybridization” refers to the ability of a first polynucleotide to form at least one hydrogen bond with at least one second polynucleotide under low stringency conditions.

The term “aptamer” refers to a strand of nucleic acids that undergoes a conformational change when associated with an effector.

The term “conformational change” refers to the process by which an aptamer or DNA duplex adopts a tertiary structure from another state. For simplicity, the term “fold” may be substituted for conformational change.

The term “effector” refers to any ion or molecule that activates an error-correction enzyme or causes an aptamer to fold. Preferable monovalent ions having a ⁺1 formal oxidation state (I) include NH₄⁺, K(I), Li(I), TI(I), and Ag(I). Preferable divalent metal ions having a ⁺2 formal oxidation state (II) include Mg(II), Ca(II), Mn(II), Co(II), Ni(II), Zn(II), Cd(II), Cu(II), Pb(II), Hg(II), Pt(II), Ra(II), Sr(II), Ni(II), and Ba(II). Preferable trivalent and higher metal ions having ⁺3 (III), ⁺4 (IV), ⁺5 (V), or ⁺6 (VI) formal oxidation states include Co(III), Cr(III), Ce(IV), As(V), U(VI), Cr(VI), and lanthanide ions. Preferable biomolecules include large biomolecules, such as proteins (e.g. proteins related to HIV, hCG-hormone, insulin), oligonucleotides, antibodies, growth factors, enzymes, virus (e.g. HIV, small pox), viral derived components (e.g. HIV-derived molecules), bacteria (e.g. anthrax), bacteria derived molecules and components (e.g. anthrax derived molecules), or cells. Preferable biomolecules also may include small biomolecules, such as amino acids (e.g. arginine), nucleotides (e.g. ATP, GTP), neurotransmitters (e.g. dopamine), cofactors (e.g. biotin), peptides, or amino-glycosides. Preferable organic molecules include drugs, such as antibiotics and theophylline, or controlled substances, such as cocaine, dyes, oligosaccharides, polysaccharides, glucose, nitrogen fertilizers, pesticides, dioxins, phenols, 2,4-dichlorophenoxyacetic acid, nerve gases, trinitrotoluene (TNT), or dinitrotoluene (DNT).

### BRIEF DESCRIPTION OF THE DRAWINGS

The invention can be better understood with reference to the following drawings and description. The components in the figures are not necessarily to scale and are not intended to accurately represent molecules or their interactions, emphasis instead being placed upon illustrating the principles of the invention.

FIG. 1 represents a biological example of proof-reading and error removal during and after self-assembly.

FIG. 2A illustrates proofreading and error correction adapted to material science.

FIG. 2B represents an implementation of the proofreading and error-correction concept of FIG. 2A where DNAzyme cleavage corrects the nanostructure.

FIG. 2C represents an implementation of the proofreading and error-correction concept of FIG. 2A where nuclease cleavage corrects the nanostructure.

FIG. 2D represents an implementation of the proofreading and error-correction concept of FIG. 2A where aptamer dissociation corrects the nanostructure.

FIG. 2E plots the temperature dependent dissociation (melting curve) for nanoparticle units having different extension lengths in the presence of the effector.

FIG. 2F represents the selective error correction that may be performed due to the temperature dependent dissociation of self-assembly units having different extension length and/or base composition.

FIG. 3 represents an implementation of proofreading/error-correction in analyte biosensors formed from self-assembled nanoparticle aggregates.

FIG. 4A represents a system capable of self-assembling, proofreading, error correcting, and reassembling nanostructure materials that incorporate nanoparticles and biopolymers.

FIG. 4B shows the UV-vis spectra of a control sample including equal parts of units A and B with no error units B′ before and after proofreading/error-correction.

FIG. 4C shows the UV-vis spectra of a sample including equal parts of units A and error units B′ with no units B before and after proofreading/error-correction.

FIG. 4D shows the increase in the extinction ratio with increasing fractions of error units B′.

FIG. 4E depicts the time-dependent change in the extinction spectra when units A and B′ were allowed to self-assemble.

FIG. 4F shows the kinetics for FIG. 4E.

FIG. 4G shows that the percentage of error units B′ removed from the nanostructure increased with increasing number of error units B′ present in the structure.

FIG. 4H is an extinction ratio plot establishing that inactive DNAzyme does not bring about a change in extinction ratios.

FIG. 41 established that less than 10% of error units B′ were released in the washing and handling process in the absence of the active DNAzyme.

FIGS. 5A-5D are transmission electron microscopy (TEM) images of self-assembled nanostructure assemblies before and after proofreading/error-correction. The images were acquired with a Philips CM200 TEM. The scale bars in the insets and in the main figures are 100 nm and 50 nm, respectively.

FIG. 6A represents a system capable of self-assembling, proofreading, removing errors, and reassembling nanostructure materials that incorporate nanoparticles and biopolymers.

FIGS. 6B-6F demonstrate the temperature dependent disaggregation of the system of FIG. 6A when from 0 to 12 adenosine bases are added to the self-assembly unit.

### DETAILED DESCRIPTION

The present invention makes use of the discovery that proofreading and error-correction techniques common in biological systems may be adapted to material science. Enzymatic catalysts or aptamers may be adapted to locate and remove errors from self-assembled nanostructures. For example, DNAzymes may be used to locate and remove errors in nanostructures formed from the self-assembly of DNA-templated gold nanoparticles

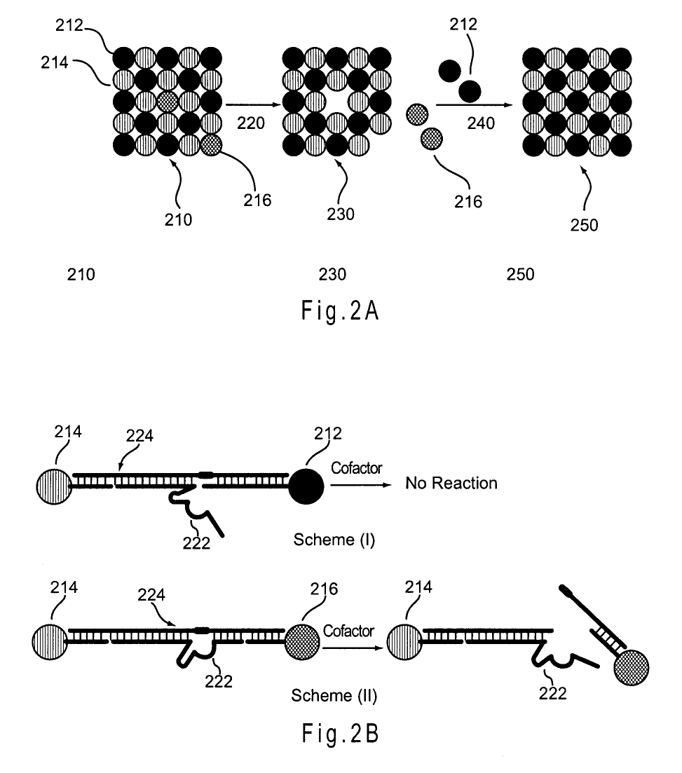

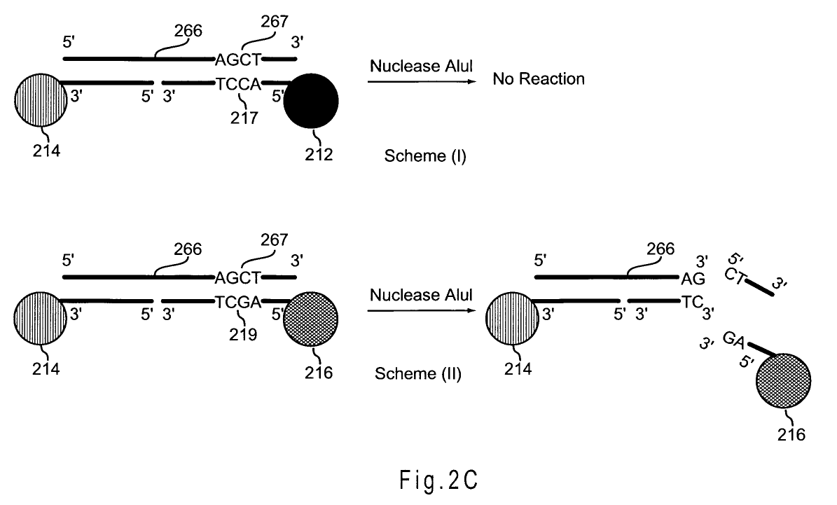

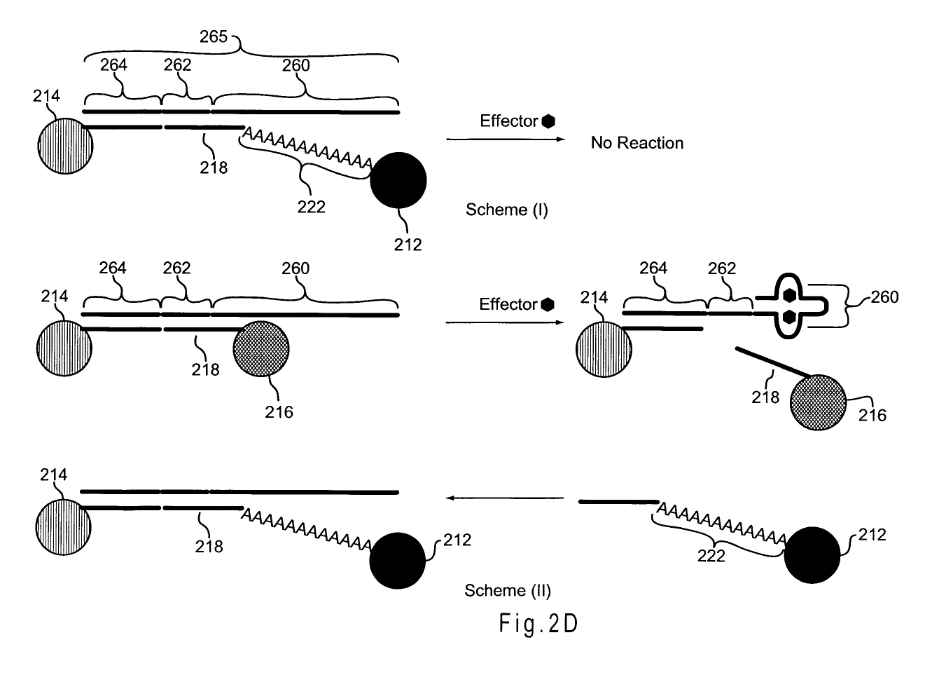

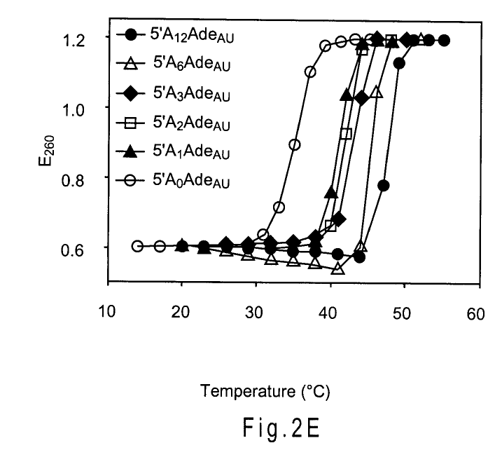

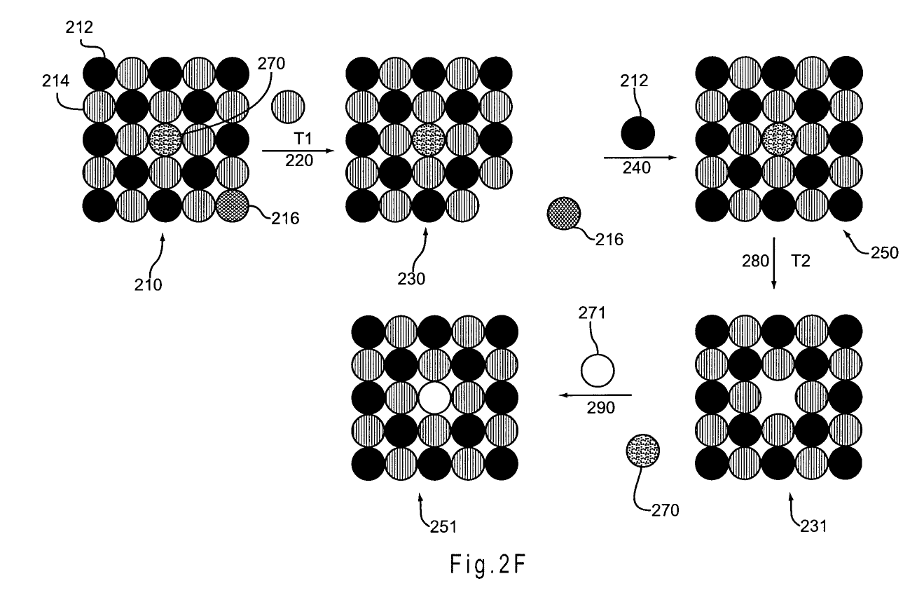

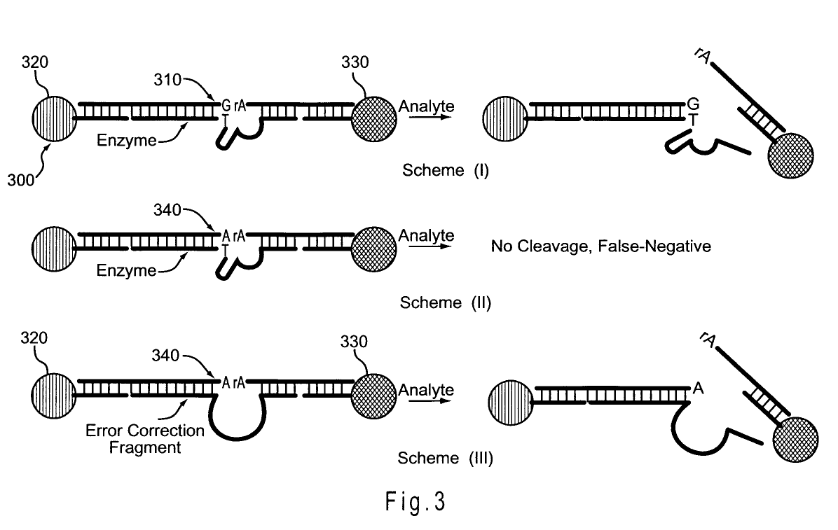

FIG. 2A illustrates the concept of biological proofreading and error correction adapted to nanomaterial synthesis. A self-assembled, nanomaterial structure 210 should include only appropriate first units 212 and second units 214. However, due to errors present in the self-assembly process, error units 216 also are incorporated. During proofreading/error-correction 220 the error units 216 are identified during proofreading and removed during correction to give a corrected nanomaterial structure 230 having voids where the error units 216 were removed.

If additional first and/or second units are present, the appropriate units may fill the voids in the corrected structure 230 during self-reassembly 240 to give a reassembled structure 250. Because the probability is greater that any unit assembled will be an appropriate unit as opposed to an error unit, one reassembly may be sufficient to provide a substantially error-free product structure, such as the reassembled structure 250. However, multiple proofreading/error-corrections and reassemblies may occur, such as when proofreading/error-correction is used during self-assembly.

The units, which form the nanostructures, may include any material that is inherently or chemically adaptable to undergo self-assembly and that is compatible with the proofreading/error-correction process. The units may include a building block in combination with an oligonucleotide. The units or building blocks may include DNA tiles, such as those containing double-crossover DNA structures. In another aspect, the building blocks may include proteins, such as strepavidin; polymers, such as polystyrene; fullerenes, such as C₆₀ or C₇₀; nanotubes; nanowires; nanoribbons; metallic nanoparticles, such as particles of Au, Ag, Cu, Pt, or Pd; magnetic nanoparticles, such as Fe₃O₄, Co, NiCo, or FeCo; semiconducting nanoparticles, such as ZnO, ZnS, ZnSe, ZnTe, CdO, CdS, CdSe, CdTe, PbSe, GaN, Gain, SiO₂, or Si; quantum dots; or any combination thereof. The bonding that occurs between the units during self-assembly or self-reassembly may be any type of bonding including covalent, dative, ionic, hydrogen, van der Waals, or any combination thereof.

The proofreading/error-correction 220 may be performed by a single or multiple error correcting unit (ECU). The ECU that proofreads the structure 210 and perform the error correction may include DNAzymes, RNAzymes, protein enzymes, proteins, nucleic acids, aptamers, carbohydrates, peptide nucleic acids, biomimetic polymers, organic molecules having a molecular weight of less than 1500, organic macromolcules, or any combination thereof. In a preferred aspect, the proofreading and error-correction functions of the ECU are provided by a single enzyme that catalyzes cleavage or ligation. In another preferred aspect, the proofreading and error-correction functions of the ECU are provided by an aptamer. While DNAzymes and RNAzymes may perform these functions, DNAzymes may be preferred because they are readily made and stable. In another preferred aspect, the proofreading and error-correction functions are of the ECU are provided by an aptamer strand that folds.

FIG. 2B represents a proofreading and error-correction cleavage process, such as represented by the proofreading/error-correction 220 of FIG. 2A. In Scheme (I), the appropriate self-assembly units 214, 212 self-assemble to a cleavable substrate 224. In Scheme (II) the appropriate self-assembly unit 214 and the error unit 216 self-assemble to the cleavable substrate 224.

Each of the self-assembly units includes a nanoparticle functionalized with a different DNA strand. The DNA strand on the appropriate unit 212 is longer than the DNA strand of the error unit 216. In Scheme (I) the longer DNA strand of the unit 212 may bind to the cleavable substrate 224 farther along the length of the substrate 224. In this manner, an enzyme 222 may be prevented from attaining a catalytically active conformation capable of cleaving the substrate 224.

Conversely, in Scheme (II), the shorter DNA strand of the error unit 216 allows the enzyme 222 to attain the catalytically active conformation and cleave the substrate 224. When an appropriate co-factor is supplied to the enzyme, the enzyme 222 cleaves the error unit 216 from the unit 214. Thus, the error unit 216 may be removed by the catalytic function of the enzyme 222, such as a DNAzyme, while the appropriate Scheme (I) nanostructure remains intact.

While not shown in the figure, the un-assembled unit 214 having the enzyme 222 attached may now reassemble with the appropriate unit 212 to form the nanostructure of Scheme (I). In one aspect, the reassembly includes removal of the cleaved portion of the substrate 224. In another aspect, the reassembly includes replacement of the cleaved portion of the substrate 224 with the substrate 224. In another aspect, the system may be modified so that cleavage results in the enzyme 222, the substrate 224, and the error unit 216 leaving the appropriate unit 214 anchored in the nanostructure, thus permitting reassembly with the appropriate unit 212 in combination with the substrate 224 and the enzyme 222.

FIG. 2C represents another proofreading and error-correction cleavage process, such as represented by the proofreading/error-correction 220 of FIG. 2A, where a protein-based enzyme or nuclease is substituted for a DNAzyme. In Scheme (I), the appropriate self-assembly units 214, 212 self-assemble to a substrate strand 266. In Scheme (II) the appropriate self-assembly unit 214 and the error unit 216 self-assemble to the substrate strand 266. In this case, the appropriate self-assembly unit 212 includes a portion 217 that is not complementary to a portion 267 of the substrate strand 266. Conversely, the error unit 216 includes a complementary portion 219 that is complementary to the portion 267 of the substrate strand 266.

As represented in Scheme (I) of FIG. 2C, in the presence of a nuclease, such as Alul, the appropriate self-assembly unit 212 remains hybridized to the substrate strand 266. As depicted in Scheme (II), in the presence of the nuclease, the error unit 216 and a portion of the substrate strand 266 are cleaved between the C and G bases due to the complementary nature of the error unit portion 219 and the substrate strand portion 267. Nucleases generally only cleave fully complementary strands. Thus, the error unit 216 may be removed by the catalytic function of a nuclease, such as Alul, while the appropriate Scheme (I) nanostructure remains intact.

FIG. 2D represents a proofreading and error-correction dissociation process, such as represented by the proofreading/error-correction 220 of FIG. 2A. In Scheme (I) of FIG. 2D, the appropriate self-assembly units 214, 212 self-assemble to a substrate 265. In Scheme (II) the appropriate self-assembly unit 214 and the error unit 216 self-assemble to the substrate 265. The substrate 265 may include an aptamer portion 260, an overhang portion 264 for hybridizing with the appropriate self-assembly unit 214, and a linker portion 262 joining the aptamer 260 and the overhang 264. The error unit 216 may include a short DNA strand 218 that hybridizes with the linker 262 and optionally a portion of the aptamer 260. In addition to the short DNA strand 218 of the error unit 216, the appropriate self-assembly unit 214 may include an extension 222 separating the nanoparticle from the short DNA strand 218.

Each of the self-assembly units includes a nanoparticle functionalized with a different DNA strand. The DNA strand of the appropriate unit 212 is longer by the 12 adenosine units of the extension 222 than the DNA strand of the error unit 216. In Scheme (II), the aptamer portion 260 of the substrate 265 folds in the presence of an effector for the aptamer 260. This folding dissociates the error unit 216. Thus, the error unit 214 may be removed by the folding of the aptamer 260, while the appropriate unit 212 remains intact as depicted in Scheme (I). The length and base composition of the extension 222 may be altered to control the ease of folding by the aptamer 260 in the presence of the effector. As also depicted in Scheme (II), if another appropriate self-assembly unit 212 is present after the error-correction, the appropriate self-assembly unit 212 may hybridize with the substrate 265 to form the nanostructure of Scheme (I).

In another aspect, the aptamer portion 260 may be substituted with two hybridized strands, such as a DNA duplex. In this system, the presence of the appropriate effector induces a major-minor groove conformational change in the DNA duplex, thus dissociating the error unit 216. As in the aptamer system, the appropriate self-assembly unit 212 may then hybridize with the substrate 265 to form the nanostructure of Scheme (I).

FIG. 2E shows the temperature dependent dissociation (melting curve) for nanoparticle units having different extension lengths in the presence of the effector. When the extension 222 has 0 adenosine bases, the error unit 216 of FIG. 2D may be provided. When the extension 222 has 12 adenosine bases, the appropriate unit 212 of FIG. 2D may be provided. When the length of the extension 222 increases from 1 to 11 bases, the self-assembly unit transitions from the error unit 216 to the appropriate unit 212. As shown in the figure, the dissociation, and thus error behavior, of a specific self-assembly unit is temperature dependent. Thus, a self-assembly unit having 2 extension bases serves as the appropriate unit 212 at temperatures less than about 40° C. and as the error unit 216 at temperatures greater than about 40° C.

FIG. 2F represents the selective error correction that may be performed due to the temperature dependent dissociation of self-assembly units having different extension length and/or base composition. A self-assembled, nanomaterial structure 210 should include only first units 212 and second units 214. However, due to errors present in the self-assembly process, error units 216 and 270 also may be incorporated. In comparison to the nanomaterial structure 210 of FIG. 2A, the FIG. 2F structure includes two different error units 216, 270.

When the effector for the aptamer is added during the proofreading/error-correction 220 at T1, the error units 216 are identified during proofreading and removed during correction to give the corrected nanomaterial structure 230 having voids where the error units 216 are removed. If additional first and/or second units are present, the appropriate units may fill the voids in the corrected structure 230 during self-reassembly 240 to give the reassembled structure 250. However, the error units 270 remain in the reassembled structure 250. By then changing the temperature to T2 in 280, the error units 270 are identified during proofreading and removed during correction to give corrected nanomaterial structure 231 having voids where the error units 270 are removed. If additional first and/or second units (not shown), or optional third appropriate units 271 are present, the third appropriate units 271 may fill the voids in the corrected structure 231 during self-reassembly 290 to give the reassembled structure 251. In this manner, errors may be selectively corrected and replaced in a nanomaterial structure. Furthermore, appropriate units that are incompatible with a single self-assembly process may be incorporated into the structure due to the ability to selectively remove incorporated units at different temperatures.

Because self-assembled nanostructures may be used in many applications, the proofreading/error-correction of the present invention has many applications. Such applications may include improving the performance of analyte sensors, improving DNA computing circuits, improving the assembly of photonic crystals, and improving the quality of pixilated displays.

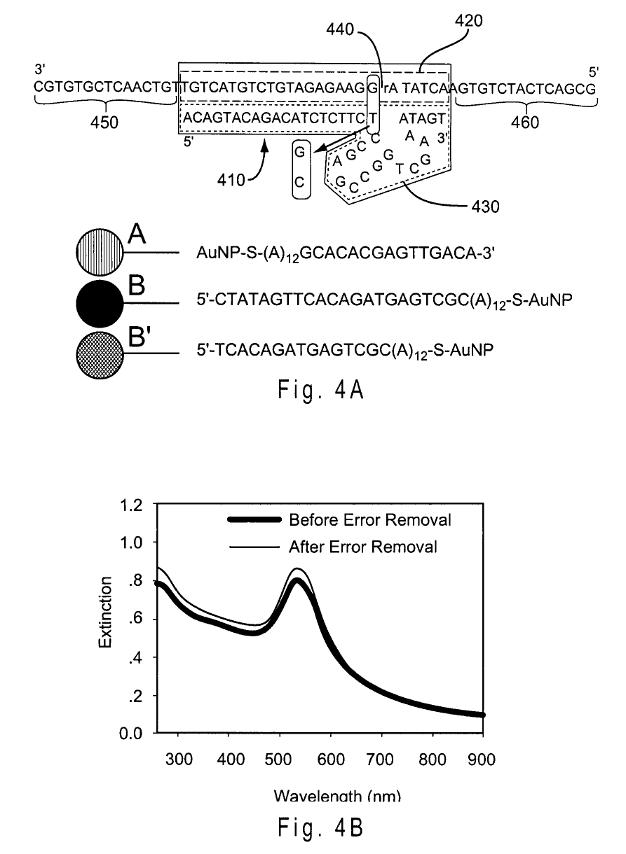

DNA-templated nanoparticle assembly may provide the desired resolution, controllability, and versatility during self-assembly to provide useful nanostructures. One useful implementation of DNA-templated nanoparticle assembly is in the context of analyte sensors.¹¹⁻¹⁹ FIG. 3 represents a cleavage-type implementation of proofreading/error-correction in analyte biosensors formed from self-assembled nanoparticle aggregates. In the same way that individual amino acids are coded by specific tRNA's, DNA-templated nanoparticle sensor assemblies may be formed where each nanoparticle is coded by a DNA molecule having a unique sequence.

As shown in Scheme (I) of FIG. 3, a sensor 300 normally operates by the self-assembly of a cleavable substrate 310, which holds nanoparticles 320 and 330 in close proximity. In the presence of the selected analyte, the substrate is cleaved by an enzyme. When the nanoparticles 320 and 330 separate after cleavage of the substrate, they move farther apart and undergo a color change to signify the presence of the analyte.

However, as depicted in Scheme (II) of FIG. 3, when an incorrect substrate for the enzyme 340 is formed from an error in the self-assembly of the nanostructure aggregate, such as a mutation from a G to an A base, the cleavage reaction does not occur in the presence of the analyte. Thus, even though the analyte is present, a color change does not occur. This results in a false-negative result for the analysis.

In Scheme (III) of FIG. 3, the analyte sensor of Scheme (II) is modified in accord with the present invention to include a proofreading/error-correction fragment. In this manner, the incorrect substrate 340 is cleaved, thus removing the nanoparticles joined by errors in the initial self-assembly of the nanostructure and providing the correct positive analysis result for the analyte.

In another aspect, proofreading/error-correction may be used to improve DNA computing circuits. Self-assembled circuit patterns using DNA tiles are a leading option for DNA computing circuits. DNA tiling systems are arrangements of multi-strand DNA structures having unpaired extensions from a paired helix. A single tile unit may include DNA structures that may be double, triple, or quad stranded and also may include 3- or 4-way junctions in combination with multiple “sticky ends.” The sticky ends permit self-assembly through hybridization of the tiles.

However, with self-assembly comes the inherent creation and propagation of errors. Errors arise from undesired partially complimentarity hybridizations leading to incorrect tile assembly. The proofreading/error-correction systems of the present invention may correct improperly self-assembled DNA tiles by binding termination. For example, if tile A binding to tile B is desired and strong, while tile A binding to tile C is undesired and weak, the error correction fragment with destroy the A-C binding, such as by truncation or ligation. The incorrect A-C binding may then be replaced with the stronger A-B tile binding.

In another aspect, proofreading/error-correction may be used to improve the assembly of photonic crystals. When forming photonic crystals, three average diameters may be used for the nanoparticles. Only two of these average diameters are desired for the nanoparticles in the final product, while the nanoparticles having the third average diameter is used to facilitate formation of the desired packing structure for the desired nanostructure. Using a proofreading/error-correction system, the third particles may be removed from the photonic crystal after formation.

In another aspect, proofreading/error-correction may be used to remove undesired pixels from self-assembled displays. When a pixilated display requires four sub-pixels, such as a red, a blue, and two green sub-pixels, to form an image, some of the pixels will be faulty. For example, a pixel may include a blue, two red, and only one green sub-pixel. The implementation of proofreading/error-correction in accord with the present invention may remove the extra red sub-pixel and permit self-reassembly to provide the desired green sub-pixel.

The following examples are provided to illustrate one or more embodiments of the invention. Numerous variations can be made to the following examples that lie within the scope of the invention.

### EXAMPLES

### Example 1

### DNAzyme Proofreading and Error Correction

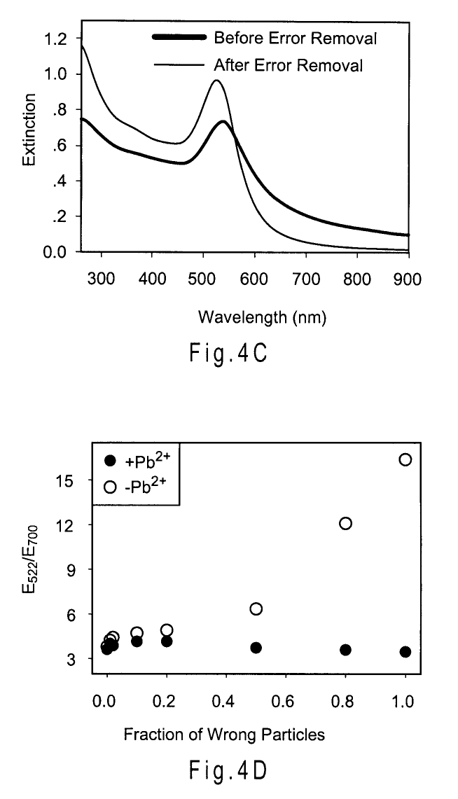

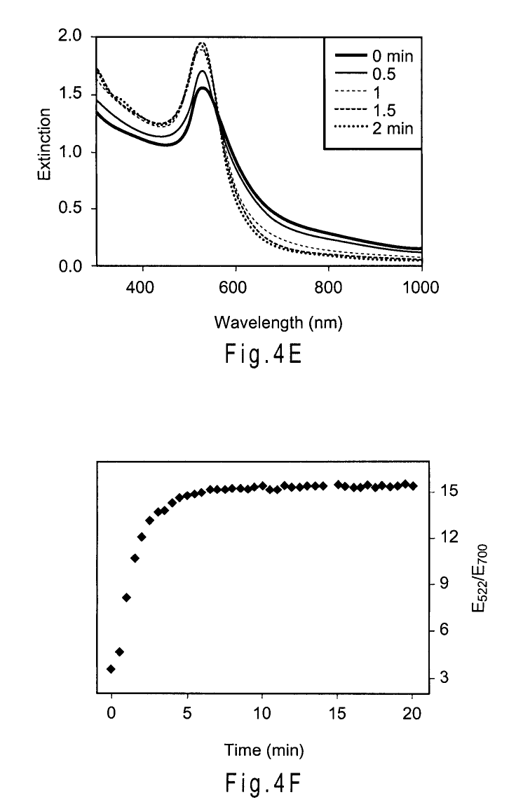

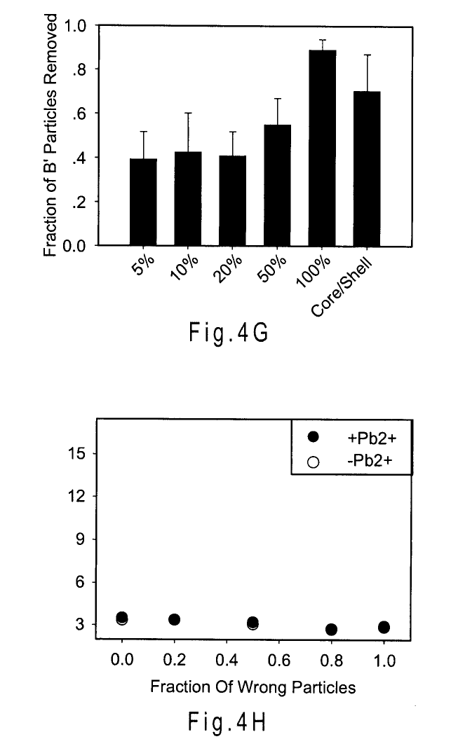

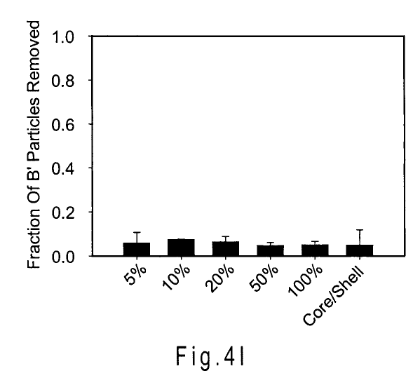

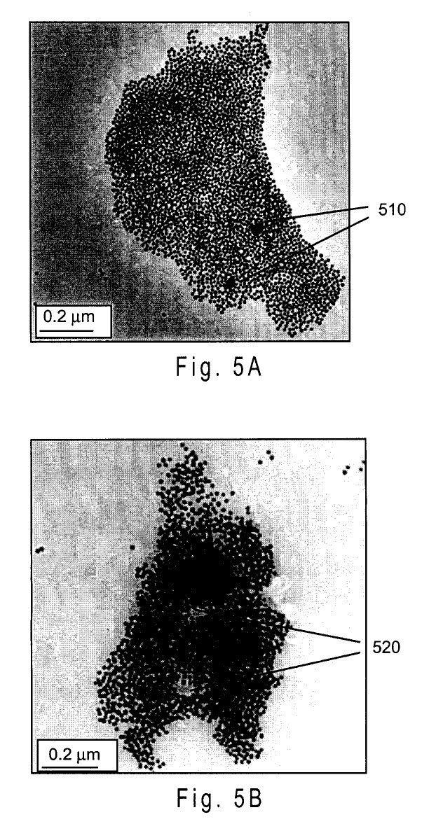

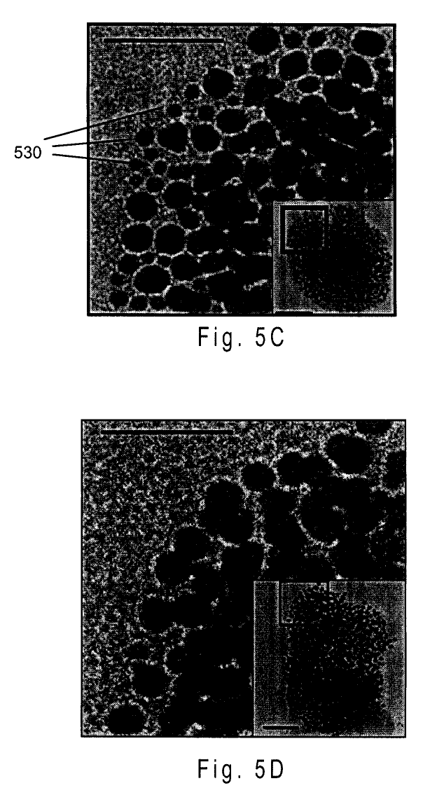

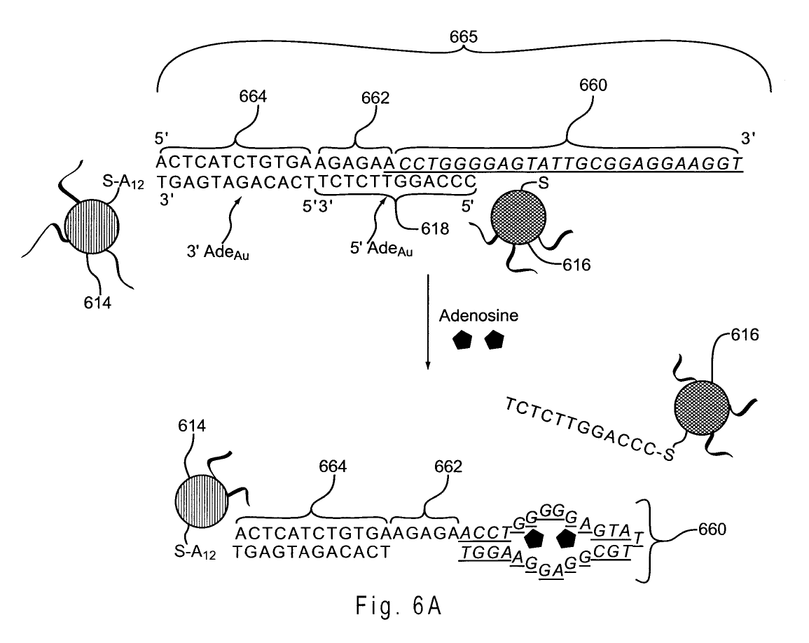

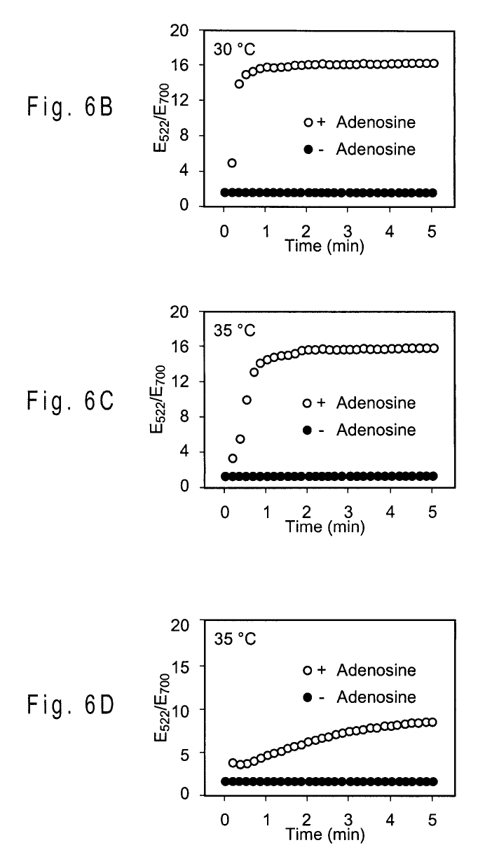

FIG. 4A represents a system capable of self-assembling, proofreading, removing errors, and reassembling nanostructure materials that incorporate nanoparticles and biopolymers. Thus, the system of FIG. 4A is one example of the proofreading/error-correction concept described above with regards to FIG. 2B.

A DNAzyme, such as a DNAzyme 410 having the sequence depicted in FIG. 4A, may be used for nanostructure synthesis. Unlike their biological counterparts, DNAzymes may perform both a synthesis function during the self-assembly of the nanoparticles and the proofreading/error-correction function.²⁰⁻²² The DNAzyme 410 may include a substrate strand 420 and an enzyme strand 430 that form two duplex regions on either side of a cleavage site 440.

In the presence of a co-factor, such as Pb²⁺ for the specific DNAzyme 410, the substrate strand 420 may be cleaved into two pieces at the rA position by the DNAzyme 410. The two duplex regions may be designed to be unsymmetrical with the left side including 19 base pairs, while the right side may include 5 base pairs.

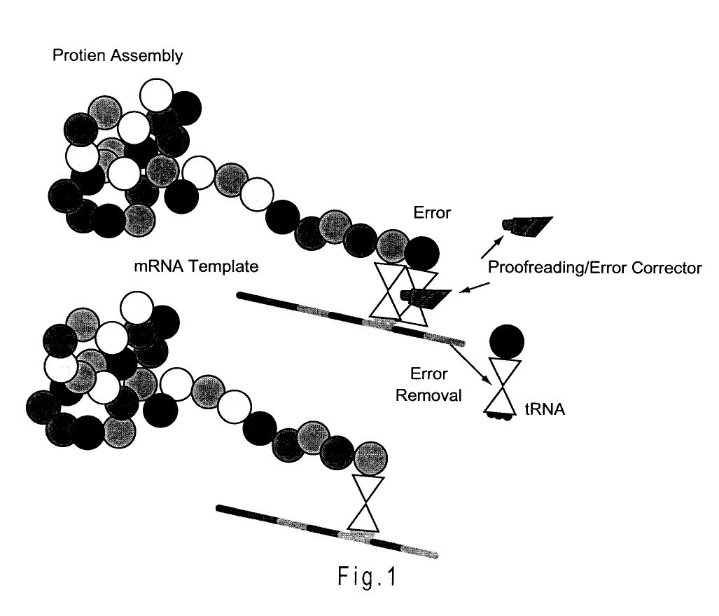

To allow the substrate strand 420 to serve as a template for nanoparticle assembly, the overhangs 450 and 460 on the substrate strand 420 may be complementary to the DNA strands attached to the self-assembly units A and B, as discussed below. In one aspect, the DNAzyme 410 may be considered to have functionality similar to the protein enzymes used for proof-reading and error removal during the biological protein synthesis of FIG. 1.

Units for self-assembly by the DNAzyme 410 were prepared by functionalizing 13 nm average diameter gold nanoparticles with three different DNA strands. The resulting units for self-assembly are depicted in FIG. 4A as A, B, and B′, with B′ being the error unit. The DNA sequences for the specific DNA strands attached to the gold nanoparticles (AuNP) for this example also are shown in FIG. 4A. The (A)₁₂ portion of the sequence denotes a poly A spacer containing 12 A bases.

Unit A can bind to the 3′ end of the DNA template 450, while unit B binds to the 5′ end of the template 460, both through sequence-specific hybridization. The error unit B′ also may bind to the 5′ end of the template 460.

However, the DNA attached to the error unit B′ is 7-bases shorter than the DNA attached to unit B. As a result, the affinity of the error unit B′ to the template is less than that of unit B to the template. In one aspect, this decreased affinity of the error unit B′ to the DNA template may be considered similar to the decreased affinity between an error tRNA and the mRNA template, as previously described with regard to the biological system of FIG. 1.

If a correct unit B is incorporated, binding of the longer arm of the enzyme strand 430 to the substrate strand 420 is permitted while binding of the shorter arm to the DNA template is inhibited, and the active structure of the DNAzyme 410 does not form. As a result, the substrate strand 420 is not cleaved and the unit B is incorporated into the nanostructure. When an error unit B′ is incorporated into the nanostructure, the enzyme strand 430 may bind both of its duplex regions to the substrate strand 420. This double binding forms an active catalyst in the presence of a co-factor that cleaves the substrate strand 420 and removes the error unit B′.

To establish the specific removal of error units B′ from a nanostructure, A, B, and B′ units were combined and allowed to self-assemble. FIG. 4B shows the UV-vis spectra of a control sample including equal parts of units A and B with no error units before and after proofreading/error-correction. Only a slight increase in the extinction coefficient at the 522 nm peak was observed after the proofreading/error-correction process was initiated with an appropriate co-factor. The lack of a significant change in the extinction coefficient established that the self-assembled nanostructure lacking error units did not significantly change during the proofreading/error-correction process.

FIG. 4C shows the UV-vis spectra of a control sample including equal parts of units A and error units B′ with no units B before and after proofreading/error-correction. A small increase was observed at the 522 nm peak, while a substantial decrease was observed at the 700 nm peak after the correction process. The resulting high extinction ratio of ˜>15 indicated that the units A and B′ in the nanostructure were disassembled.^{18,19} Together, FIGS. 4B and 4C establish the extremes for the proofreading/error-correction process.

To demonstrate the effectiveness of the proofreading/error-correction process at correction levels between the extremes of FIGS. 4B and 4C, A, B, and B′ units were initially mixed so that the number of B units equaled the sum of the A and B′ error units. As seen in FIG. 4D, when the fraction of error units was increased, the extinction ratio increased in the presence of the Pb²⁺ co-factor.

FIG. 4E depicts the time-dependent change in the extinction spectra when all units were A and B′. After ˜2 minutes, the change in the spectra was insignificant, suggesting that the kinetics were fast. FIG. 4F depicts the kinetics of the process as determined by monitoring the extinction ratio for twenty minutes. These kinetic experiments establish that the error correction process is rapid.

### Example 2

### Determining Percent Correction

To estimate the percent of error correction occurring in a self-assembled nanostructure, error units B′ were functionalized with fluorescein-labeled DNA in addition to the original fluorescein-free DNA. After adding the Pb²⁺ co-factor, the samples were centrifuged at 2000 rpm for 2 minutes. The assembled nanostructures centrifuged to the bottom, while the dispersed particles remained in the supernatant. After the supernatant was removed, the precipitate was re-dispersed and re-centrifuged twice to completely separate self-assembled nanostructures from the dispersed units.

The fluorescein-labeled DNA was then displaced into solution by adding a high concentration of small alkylthiol molecules, such as β-mercaptopropionic acid.²⁴ By comparing the fluorescence intensity from the self-assembled nanostructures with the fluorescence intensity of the supernatant, the percentage of error units B′ released from the assembled nanostructures was calculated. As shown in FIG. 4G, the percentage of error unit B′ removal increased with the percentage of error units B′ present in the assembled nanostructures, from ˜40% removal at low percentage of B′ to almost complete removal at 100% B′ level.

The incomplete removal of error units B′ at low percentages may be attributed to error units B′ embedded in the interior of the nanostructure, which may not be released even if the DNA template was cleaved. To support this hypothesis, assembled nanostructures made of A and B units were prepared as a core nanostructure. Error units B′ were added to form an external shell for the core to ensure that the error units were exposed to the surface of the assembly. Indeed, at similarly low percentage of B′, the percent removal of B′ increased from ˜40% to ˜70% for the core-shell self-assembled nanostructure.

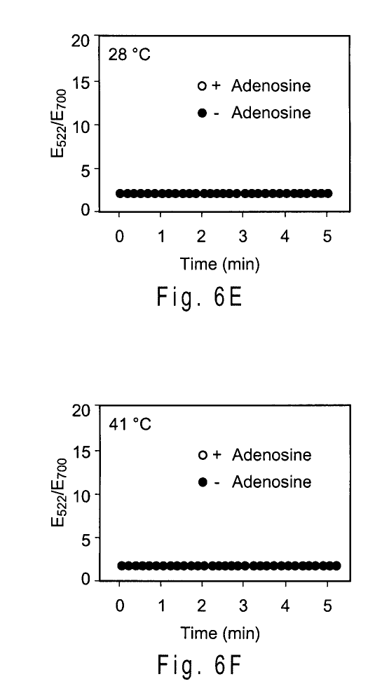

The non-specific release of units could occur during the proofreading/self-assembly process, which would contribute to the calculated percentage of error removal. To investigate this possibility, a control experiment was performed with an inactive DNAzyme, which had the same structure as the active DNAzyme except that one base was mutated (a T base was mutated to a C base, as depicted in FIG. 4A).²³ FIG. 4H established that a change was not observed in the extinction ratio of the nanostructures with the inactive DNAzyme. Additionally, FIG. 41 established that less than 10% of error units B′ were released in the washing and handling process in the absence of the active DNAzyme. Thus, non-specific dissociations cannot account for the observed error removal.

### Example 3

### Transmission Electron Microscopy Images

To further confirm that the error units were removed from the nanostructures, the average diameter of the error units was increased from 13 nm to 50 nm. About 1% error units were mixed with about 99% first units A (13 nm) and second units B (13 nm). FIG. 5A established that two error units 510 were incorporated in the nanostructure aggregate after the initial self-assembly. After adding a Pb²⁺ co-factor to activate the correction enzyme, FIG. 5B established that voids 520 were created in the nanostructure. Thus, the error units 510 were removed from the self-assembled nanostructure of FIG. 5A through proofreading/error-correction.

FIG. 5C depicts a nanostructure where 13 nm first A and second B units were combined with 5 nm error units B′. Smaller error units 530 are visible in FIG. 5C. After adding a Pb²⁺ co-factor to activate the correction enzyme, FIG. 5D established that the smaller error units 530 have been substantially removed from the nanoparticle structure.

### Example 4

### Nanoparticle Preparation and Functionalization

Gold nanoparticles of 13 nm diameter were prepared with the citrate reduction method.²⁵ Gold nanoparticles of 5 nm diameter were prepared with the NaBH₄ reduction method.²⁶ Thiol-labeled DNA molecules were attached to gold nanoparticles by literature methods.²⁵ Nanoparticles were purified twice by centrifugation, removal of the supernatant, and re-suspension in new buffer (100 mM NaCI, 25 mM Tris acetate, pH 8.2). To prepare fluorescein-labeled error units B′, in addition to the original DNA (80%), 5′-FAM-(A)₁₂-SH-3′ (20%) also was added.

### Example 5

### Unit Assembly

Units A, B, and/or B′ were mixed at designated ratios to provide a final extinction coefficient at 522 nm of ˜2. The system also contained 100 nM of the substrate, 200 nM of the enzyme strand (or the mutated enzyme), 300 mM of NaCI and 25 mM of Tris acetate, pH 8.2. The units were self-assembled by heating the sample at 65° C. for 1 minute in a water bath (containing 60 mL water), and subsequently cooling to room temperature for ˜1 hour. The sample was then centrifuged at 2000 rpm for 1 minute. The supernatant was removed and the assembled nanostructures were re-dispersed in 100 mM NaCI, 25 mM Tris acetate, pH 8.2. To prepare samples for the TEM experiments, the 5 nm error units B′ were added later to form an A/B unit core and having an error unit B′ shell nanostructure. Thus, the error units predominated on the surface of the self-assembled nanostructures, facilitating release and observation.

### Example 6

### Error Removal

Typically, two aliquots were taken from the self-assembled nanostructures. Pb²⁺ (final concentration 30 μM) was added to one of the aliquots to initiate the error correction process (reaction time 30 minutes), while the other aliquot was retained as a control.

### Example 7

### Aptamer Proofreading and Error Correction

FIG. 6A represents a system capable of self-assembling, proofreading, removing errors, and reassembling nanostructure materials that incorporate nanoparticles and biopolymers. Thus, the system of FIG. 6A is another example of the proofreading/error-correction concept described above with regard to FIG. 2B and an example of the selective proofreading/error-correction concept described above with regard to FIG. 2E.

The system includes a substrate 665 that includes an overhang portion 664 joined to an aptamer 660 by a linker portion 662. The base sequence of each portion of the substrate 665 is shown. The base sequence of the aptamer 660 was chosen to fold as adenosine is bound; however, any aptamer that folds in response to a specific effector that is combatable with the self-assembly, proofreading, and error correction of the nanostructure may be used. The base sequence of the overhang portion 664 was chosen to hybridize with the functionalized nanoparticle unit 614. Any base sequence may be chosen for the overhang portion 664 and the functionalized nanoparticle that allows their hybridization. The base sequence of the linker portion 662 was chosen to join the aptamer 660 to the overhang 664 and to hybridize with a second functionalized nanoparticle.

When adenosine was added to the system, functionalized nanoparticle unit 616 dissociates, thus correcting the error. This dissociation may be attributable to the depicted folding of the aptamer 660 in response to the adenosine effector. While the depicted aptamer 660 is specific to adenosine, any aptamer that undergoes a conformational change in response to an effector may be useful. A list of useful aptamers and their effectors (analytes as in Table I) may be found in U.S. patent application Ser. No. 11/202,380, filed Aug. 11, 2005; Table I of which is incorporated herein.

FIGS. 6B-6F demonstrate the temperature dependent disaggregation of the system of FIG. 6A when from 0 to 12 adenosine bases are inserted between bases 618 and the nanoparticle of the unit 616. FIG. 6B establishes that when 0 adenine bases are inserted between the bases 618 and the nanoparticle at 30° C., the addition of adenosine promotes substantially complete dissociation of the unit 616. Thus, making the unit with 0 adenine bases an error unit at 30° C. FIGS. 6C-6D establish that when 1 (FIG. 6C) or 2 (FIG. 6D) adenine bases are inserted between the bases 618 and the nanoparticle at 35° C., the addition of adenosine promotes substantially complete dissociation of the unit 616. Thus, making the unit with 1 or 2 adenine bases an error unit at 35° C. FIG. 6E establishes that when 6 adenine bases are inserted between the bases 618 and the nanoparticle at 38° C., the addition of adenosine does not dissociate the unit 616. Thus, making the unit with 6 adenine bases an appropriate unit at 38° C. Similarly, FIG. 6F establishes that when 12 adenine bases are inserted between the bases 618 and the nanoparticle at 38° C., an appropriate unit is provided.

While various embodiments of the invention are described, it will be apparent to those of ordinary skill in the art that other embodiments and implementations are possible within the scope of the invention.

### REFERENCES

1. Seeman, N. C. DNA in a material world. Nature 421, 427-431 (2003).
2. Van Blaaderen, A., Rue, R. & Wiltzius, P. Template-directed colloidal crystallization. Nature 385, 321-324 (1997).
3. Wong, G. C. L. et al. Hierarchical self-assembly of F-actin and cationic lipid complexes: Stacked three-layer tubule networks. Science 288, 2035-2039 (2000).
4. Niemeyer, C. M. Nanoparticles, proteins, and nucleic acids: biotechnology meets materials science. Angew. Chem. Int. Ed. 40, 4128-4158 (2001).
5. Katz, E. & Willner, I. Nanobiotechnology: integrated nanoparticle-biomolecule hybrid systems: Synthesis, properties, and applications. Angew. Chem. Int. Ed. 43, 6042-6108 (2004).
6. Fan, H. et al. Self-assembly of ordered, robust, three-dimensional gold nanocrystal/silica arrays. Science 304, 567-571 (2004).
7. Heath, J. R., Kuekes, P. J., Snider, G. S. & Williams, R. S. A defect-tolerant computer architecture: opportunities for nanotechnology. Science 280, 1716-1721 (1998).
8. Reif, J. H., LaBean, T. H., Sahu, S., Yan, H. & Yin, P. Design, simulation, and experimental demonstration of self-assembled DNA nanostructures and motors. Adv. Sci. Tech. 44, 311-320 (2004).
9. Roweis, S. & Winfree, E. On the reduction of errors in DNA computation. J. Comput. Biol. 6, 65-75 (1999).
10. Blanchard, S. C., Gonzalez, R. L., Kim, H. D., Chu, S. & Puglisi, J. D. tRNA selection and kinetic proofreading in translation. Nat. Struct. Mol. Biol. 11, 1008-1014 (2004).
11. Mirkin, C. A., Letsinger, R. L., Mucic, R. C. & Storhoff, J. J. A DNA-based method for rationally assembling nanoparticles into macroscopic materials. Nature 382, 607-609 (1996).
12. Alivisatos, A. P. et al. Organization of ‘nanocrystal molecules’ using DNA. Nature 382, 609-611 (1996).
13. Winfree, E., Liu, F., Wenzler, L. A. & Seeman, N. C. Design and self-assembly of two-dimensional DNA crystals. Nature 394, 539-544 (1998).
14. Yan, H., Park, S. H., Finkelstein, G., Reif, J. H. & LaBean, T. H. DNA-templated self-assembly of protein arrays and highly conductive nanowires. Science 301, 1882-1884 (2003).
15. Shih, W. M., Quispe, J. D. & Joyce, G. F. A 1.7-kilobase single-stranded DNA that folds into a nanoscale octahedron. Nature 427, 618-621 (2004).
16. Elghanian, R., Storhoff, J. J., Mucic, R. C., Letsinger, R. L. & Mirkin, C. A. Selective calorimetric detection of polynucleotides based on the distance-dependent optical properties of gold nanoparticles. Science 277, 1078-1080 (1997).
17. Rosi, N. L. & Mirkin, C. A. Nanostructures in Biodiagnostics. Chem. Rev. 105, 1547-1562 (2005).
18. Liu, J. & Lu, Y. A Colorimetric Lead Biosensor Using DNAzyme-Directed Assembly of Gold Nanoparticles. J. Am. Chem. Soc. 125, 6642-6643 (2003).
19. Liu, J. & Lu, Y. Accelerated Color Change of Gold Nanoparticles Assembled by DNAzymes for Simple and Fast Colorimetric Pb2 + Detection. J. Am. Chem. Soc. 126, 12298-12305 (2004).
20. Joyce, G. F. Directed evolution of nucleic acid enzymes. Ann. Rev. Biochem. 73, 791-836 (2004).
21. Santoro, S. W. & Joyce, G. F. A general purpose RNA-cleaving DNA enzyme. Proc. Natl. Acad. Sci. U. S. A. 94, 4262-4266 (1997).
22. Li, J., Zheng, W., Kwon, A. H. & Lu, Y. In vitro selection and characterization of a highly efficient Zn(II)-dependent RNA-cleaving deoxyribozyme. Nucleic Acids Res. 28, 481-488 (2000).
23. Brown, A. K., Li, J., Pavot, C. M. B. & Lu, Y. A Lead-Dependent DNAzyme with a Two-Step Mechanism. Biochemistry 42, 7152-7161 (2003).
24. Demers, L. M. et al. A Fluorescence-Based Method for Determining the Surface Coverage and Hybridization Efficiency of Thiol-Capped Oligonucleotides Bound to Gold Thin Films and Nanoparticles. Anal. Chem. 72, 5535-5541 (2000).
25. Storhoff, J. J., Elghanian, R., Mucic, R. C., Mirkin, C. A. & Letsinger, R. L. One-Pot Colorimetric Differentiation of Polynucleotides with Single Base Imperfections Using Gold Nanoparticle Probes. J. Am. Chem. Soc. 120, 1959-1964 (1998).
26. Hayat, M. A. (ed.) Colloidal Cold: Principles, Methods, and Applications (Academic Press, San Diego, 1991).
27. Winfree, et al. Proofreading Tile Sets: Error Correction for Algorithmic Self-Assembly, Lecture Notes in Computer Science, 2943, 126-144.
28. Nucleic Acids Research, 32,e162, (2004).

1. A self-assembled structure comprising:
(i) an error correcting unit;
(ii) a substrate;
(iii) a plurality of first appropriate units, the first appropriate units each comprising a first oligonucleotide attached to a first building block;
(iv) a plurality of second appropriate units, the second appropriate units each comprising a second oligonucleotide attached to a second building block; and
(v) a plurality of error units, the error units each comprising a third oligonucleotide attached to a third building block, where
binding of an effector to the error correcting unit causes removal of at least a portion of the error units from the self-assembled structure.

2. The structure of claim 1, where the first and second building blocks are the same.

3. The structure of claim 1, where the building blocks are particles.

4. The structure of claim 1, where the error correcting unit it selected from the group consisting of DNAzymes, RNAzymes, protein enzymes, proteins, nucleic acids, aptamers, carbohydrates, peptide nucleic acids, biomimetic polymers, organic molecules having a molecular weight of less than 1500, organic macromolcules, and combination thereof.

5. The structure of claim 1, where the error correcting unit it selected from the group consisting of DNAzymes, RNAzymes, protein enzymes, aptamers, and combinations thereof.

6. The structure of claim 1, where the substrate is a nucleic acid.

7. The structure of claim 1, where the error correcting unit cleaves the substrate to remove at least a portion of the error units from the self-assembled structure.

8. The structure of claim 1, where the error correcting unit folds to remove at least a portion of the error units from the self-assembled structure.

9. The structure of claim 1, further comprising a plurality of second error units.

10. The structure of claim 9, where at least a portion of the second error units are removed at a different temperature than the temperature at which the at least a portion of the error unit were removed from the self-assembled structure.

11. A self-assembled structure comprising:
(i) a means for correcting a self-assembled nanostructure;
(ii) a substrate;
(iii) a plurality of first appropriate units, the first appropriate units each comprising a first oligonucleotide attached to a first building block;
(iv) a plurality of second appropriate units, the second appropriate units each comprising a second oligonucleotide attached to a second building block; and
(v) a plurality of error units, the error units each comprising a third oligonucleotide attached to a third building block, where
binding of an effector to the error correcting unit causes removal of at least a portion of the error units from the self-assembled structure.

12. The composition of claim 11, where the means for correcting is selected from the group consisting of a DNAzyme, an aptamer, and a combination thereof.

13. The composition of claim 11, where the substrate is a nucleic acid.

14. The composition of claim 11, where the building blocks are particles.

15. A method for correcting errors in self-assembled nanostructures, comprising:
removing a first error unit from a self-assembled nanostructure with an error correcting unit, where
the self-assembled nanostructure comprises at least a first appropriate unit, a second appropriate unit, and the first error unit, where each unit comprises an oligonucleotide and a building block.

16. The method of claim 15, where the removing is performed by cleaving a substrate in response to a co-factor.

17. The method of claim 15, where the removing is performed by folding the error correcting unit in response to an effector.

18. The method of claim 15, further comprising reassembling the nanostructure without the first error unit.

19. The method of claim 18, further comprising removing a second error unit at a different temperature than the temperature at which the first error unit was removed.

20. The method of claim 18, where the error correcting unit it selected from the group consisting of DNAzymes, RNAzymes, protein enzymes, proteins, nucleic acids, aptamers, carbohydrates, peptide nucleic acids, biomimetic polymers, organic molecules having a molecular weight of less than 1500, organic macromolcules, and combination thereof.
